(19) United States
(12) Patent Application Publication
(10) Pub. No.: US 2015/0150455 A9
(43) Pub. Date: Jun. 4, 2015

(54) Stimulus-evoked vestibular evaluation system, method and apparatus

(71) Applicants: John M. Epley, Portland, OR (US); Jon Birck, Portland, OR (US)

(72) Inventors: John M. Epley, Portland, OR (US); Jon Birck, Portland, OR (US)

(73) Assignee: Epley Research, LLC

(21) Appl. No.: 11/449,497

(22) Filed: Jun. 7, 2006

Related U.S. Application Data
(63) Continuation-in-part of application No. 10/715,871, filed on Nov. 17, 2003, now abandoned.
Application No. 10/715,871 is a continuation-in-part of application No. 10/188,564, filed on Jul. 3, 2002, now Pat. No. 6,800,062.
(60) Provisional application No. 60/427,484, filed on Nov. 18, 2002.

(51) Int. Cl. A61B 5/00

(52) U.S. Cl. CPC A61B 5/0053; A61B 5/4023

(57) ABSTRACT

The invented apparatus for testing a subject's vestibular response to ear pressure or sound stimuli includes a headset including one or more and preferably (two) bilateral ear-worn devices such as headphones or probes, whereby each probe is configured to be inserted into a subject's ear in an operational alignment with the subject's ear canal and sealing engagement with the wall of the subject's ear canal, and a stimulus-producing mechanism operatively coupled with the headset configured to deliver defined pressure or sound stimuli to the one or more probes. The apparatus further includes one or both of 1) an ocular response analyzer operatively coupled to the headset, the ocular response analyzer measuring the subject's nystagmatic response to the stimuli by electronic, e.g. videographic, means and computerized comparison of potentially aberrational response data from the subject to recorded baseline subject data and 2) a sway response analyzer operatively coupled to the headset, the sway response analyzer including an inertial sensor of the subject's head and torso positional and orientational response to the stimuli and computerized comparison of potentially aberrational inertial data from the subject to recorded baseline inertial subject data. The invented stimulus-evoked vestibular disorder evaluation system includes a sound or aural pressure stimulus evocation apparatus presenting sound and/or air pressure to a subject's bilateral ear canals; a postural or ocular response measurement apparatus for analyzing the subject's response to a sound or pressure stimulus evoked by the sound or pressure stimulus evocation apparatus; and a computer program in the form of instructions residing in a memory and executing in a processor, the instructions configured to control the sound or aural pressure stimulus evocation apparatus in accordance with a defined protocol designed to minimize the effects of adaptation on postural response and to detect attempted malingering by the subject, and to monitor the postural or ocular response measurement apparatus in accordance with defined parameters.

### RELATED APPLICATIONS

The present application claims priority from co-pending U.S. patent application Ser. No. 10/715,871 entitled HEAD-STABILIZED MEDICAL APPARATUS, SYSTEM AND METHODOLOGY filed Nov. 17, 2003 and subject to common ownership herewith by Epley Research, LLC of Portland, Oreg., USA, the disclosure of which is incorporated herein in its entirety.

This invention was made with government support under 1R43DC006991-01 awarded by the National Institutes of Health. The government has certain rights in the invention.

### BACKGROUND OF THE INVENTION

This invention relates generally to the field of vestibular disorder diagnosis and treatment. More particularly, it concerns the semi-automated use of subject ear sound and/or pressure stimuli and subject positional stimuli and the semi-automated analysis of subject ocular (nystagmatic) and/or postural (sway) response thereto in detecting and treating vestibular disorders.

### SUMMARY OF THE INVENTION

The invented apparatus for testing a subject's vestibular response to ear pressure or sound stimuli includes a headset including one or more ear-worn devices such a headphones or probes, wherein each probe is configured to be inserted into a subject's ear in an operational alignment with the subject's ear canal and sealing engagement with the wall of the subject's ear canal, and a stimulus-producing mechanism operatively coupled with the headset configured to deliver defined pressure or sound stimuli to the one or more probes. The apparatus further includes one or both of 1) an ocular response analyzer operatively coupled to the headset, the ocular response analyzer measuring the subject's nystagmatic response to the stimuli by electronic, e.g. videographic, means and computerized comparison of potentially aberrational response data from the subject to recorded baseline subject data and 2) a sway response analyzer operatively coupled to the headset, the sway response analyzer including an inertial sensor of the subject's head and torso positional and orientational response to the stimuli and computerized comparison of potentially aberrational inertial data from the subject to recorded baseline inertial subject data. An optional destabilizing device for producing imbalance in a nominally stable subject thereby to test the subject's vestibular function and a carrying case configured and dimensioned portably to contain and transport the headset including the probes, the stimulus-producing mechanism, the ocular response analyzer and the destabilizing device therein.

In accordance with one preferred embodiment of the invention, (two) bilateral ear probes are provided so that each ear can be easily tested alternately without manual intervention or so the two ears can be tested concurrently. This bilateral ear probe feature of the invention renders the system and apparatus far easier to use, especially to analyze the subject's sway response to sound pressure level (SPL) and/or air pressure stimuli, and makes certain anti-adaptation techniques, e.g. masking, possible. It also facilitates analysis of a subject's ocular response to ear pressure stimuli, even though ocular response typically is accomplished by stimulating only one ear at a time. Furthermore, providing (two) bilateral ear probes in the same headgear greatly automates and speeds up subject vestibular function testing, since no time-consuming and distracting physical movement of a single ear probe from one subject ear to the other is required. In yet another preferred embodiment of the invention, each of the two bilateral probes includes a corresponding microphone and the apparatus further includes an ear canal seal analyzer operatively coupled with each microphone, the seal analyzer configured to detect and annunciate any substantial failure of sealing engagement between the corresponding probe and the wall of the subject's ear canal.

The invented stimulus-evoked vestibular disorder evaluation system includes a sound or aural pressure stimulus evocation apparatus coupled to one or more of a subject's bilateral ear canals; a postural or ocular response measurement apparatus for analyzing the subject's response to a sound or pressure stimulus evoked by the sound or pressure stimulus evocation apparatus; and a computer program in the form of instructions residing in a memory and executing in a processor, the instructions configured to control the sound or aural pressure stimulus evocation apparatus in accordance with a defined automatic or manual protocol designed to minimize the effects of adaptation on postural response and to detect attempts at malingering by the subject, and to monitor and/or measure and/or analyze the postural or ocular response measurement apparatus in accordance with defined operator or machine (e.g. computerized and/or databased) parameters.

Thus, the invention involves novel stimuli presentation working in concert with novel analysis methodology. Such combined features are embedded in accordance with the invention in a compact, lightweight, portable, computer-assisted or processor-based instrument.

Moreover, in accordance with one embodiment of the invention, the processor-based system is programmed automatically to adapt in real time to the subject's detected response to stimuli and automatically in real time to modify a stored test protocol based thereon, thereby nimbly and quickly to assist an operator in testing, detecting and diagnosing vestibular function.

### BRIEF DESCRIPTION OF THE DRAWINGS

FIG. 1 is a schematic system block diagram of the invented system in accordance with one embodiment of the invention.

FIG. 2 is an isometric view of the system of FIG. 1 including the headset thereof positioned on the head of a subject shown abstractly in dashed outline and the instrumentation coupled thereto.

FIG. 3 is a schematic diagram of the sound and pressure generator electronics that form a part of the system of FIG. 1.

FIG. 4 is a flowchart illustrating the invented method in accordance with one embodiment of the invention.

FIGS. 5A and 5B are schematic illustrations of screen grabs illustrating the graphical user interface (GUI) that displays a subject's ocular and postural response, respectively, to stimuli.

FIG. 6 is a flowchart illustrating the invented method in accordance with another embodiment of the invention.

### DETAILED DESCRIPTION OF THE PREFERRED EMBODIMENTS

The invented Stimulus-Evoked Vestibular Evaluation (SEVE) or Tulliometer™ System is a system for detection and analysis of stimulus-evoked (by sound or pressure) manifestations of vestibular disorders, to be utilized in the evaluation of subjects with a complaint of dizziness or imbalance. It is designed as a practical, but comprehensive, diagnostic tool for provocative testing of sound and pressure evoked vestibular phenomena (postural destabilization and abnormal ocular phenomena) found in certain inner ear conditions that cause vertigo and imbalance. Tulliometer™ is a trademark worldwide rights in which are claimed by and reserved to Epley Research LLC.

Dizziness and balance disorders are among the most common problems in US healthcare, with 6.2 million chronically affected Americans between ages forty and sixty-nine, and a majority of those over age 70 reporting balance problems, where balance-related falls account for more than half of the accidental deaths in the elderly. The most prevalent causes of dizziness and balance disorders are accepted to be of otologic origin, and a large proportion of these, once they are detected and localized, can be resolved or ameliorated by medical or surgical means. Nevertheless, current management of many of these conditions remains limited because of difficulty in diagnosis and localization. Two significant physical signs that can be critical in the diagnosis and localization of these conditions, known respectively as the “Tullio” and “Hennebert” phenomena, are evoked by presenting sound or pressure to one ear or the other (or both concurrently) and observing the subject for vestibular signs of either increased postural sway or abnormal eye movement (nystagmus).

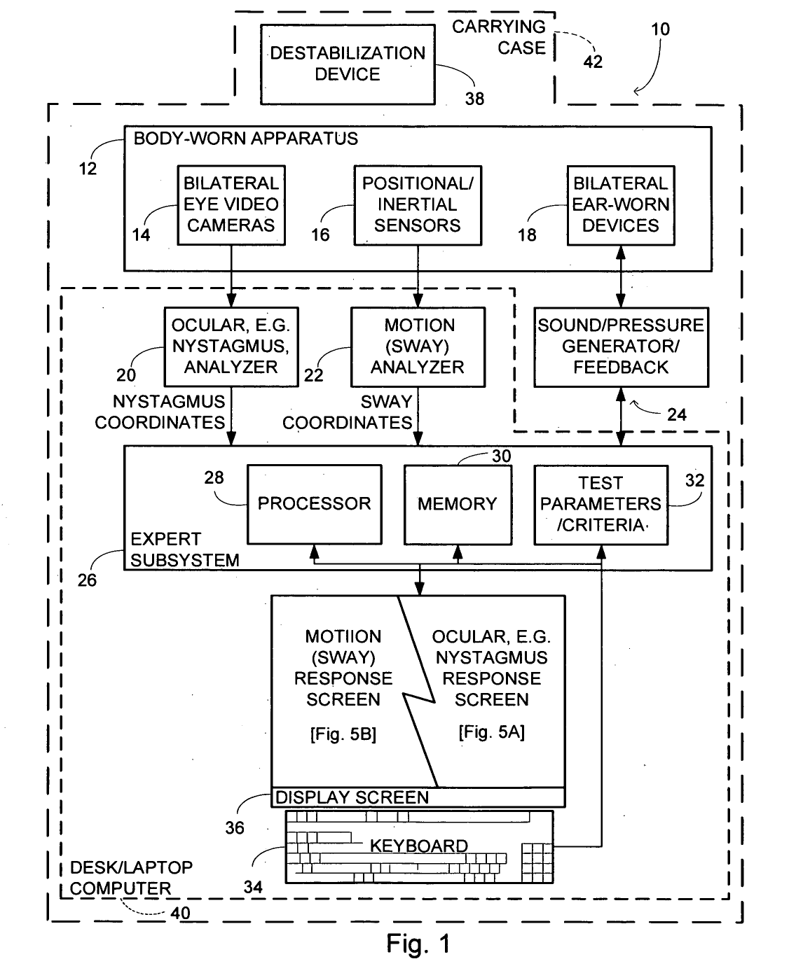

Referring to FIG. 1, the invented Stimulus-Evoked Vestibular Evaluation (SEVE) system 10 is illustrated in system block diagram form. System 10 includes body-worn apparatus 12 including bilateral eye video (e.g. infrared (IR) digital) cameras 14, one or more positional/inertial sensors 16 and bilateral ear probes 18. One or more (and preferably bilateral) ear-worn devices, e.g. ear probes 18, which can be integrated into a headset, typically are placed in position on a subject's head or torso in a comfortable position and orientation in which the ear probes sealingly engage the subject's right and/or left ear canal walls and operate to convey aural pressure or sound thereto, as will be seen. Those of skill will appreciate that, within the spirit and scope of the invention, the ear pieces instead can take the form of bilateral earphones that fit over the ears and are capable of conveying sound generated by system 10 to the subject's ears, the sound being delivered to one or more of the subject's ears in stereo (wherein two potentially different sound signals are presented to each ear) or alternatively in monaural form (wherein the same sound signals are presented to both ears). While pressure typically cannot be delivered to the subject's ears without the sealing engagement therewith as taught herein regarding ear probes 18, nevertheless sound can be productively delivered with a pair of earphones to provide sound stimulation for certain subject vestibular response testing.

One or more (and preferably bilateral) eye video cameras 14, which also can be integrated into the headset, are configured to digitally record a video of the subject's right and/or left ocular, e.g. nystagmatic, response to the ear pressure and/or sound stimuli. One or more positional/inertial sensors 16, which can take the form of an angular or gravitational accelerometer and which typically is positioned on the subject's torso such as a shoulder but which can alternatively be positioned on the subject's head, are used in accordance with the invention to monitor the subject's head or torso postural sway response to the stimuli.

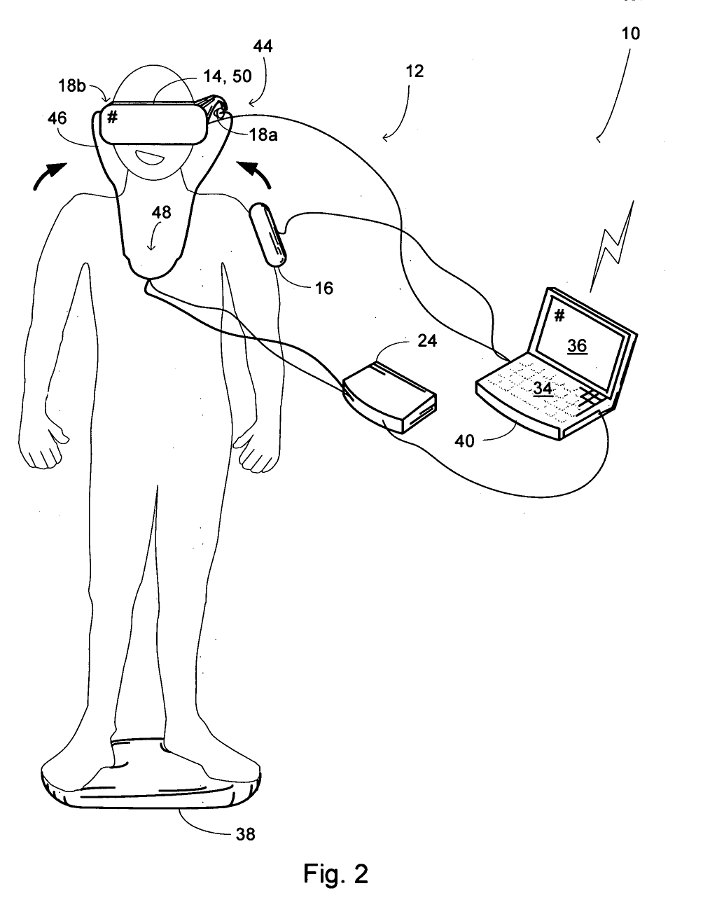

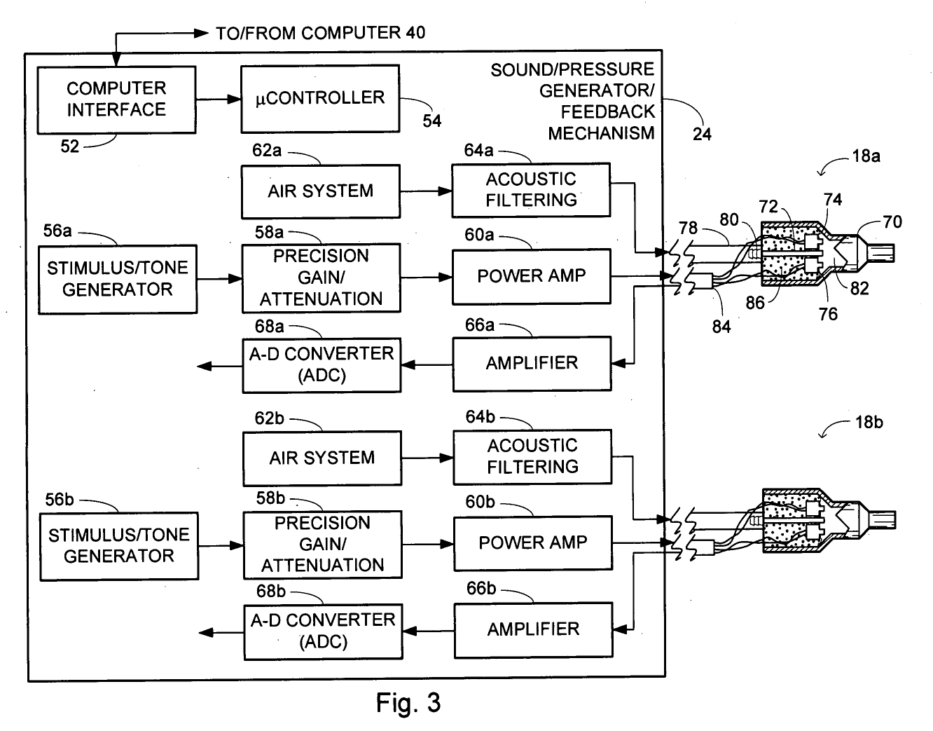

System 10 further includes an ocular analyzer, e.g. nystagmus analyzer 20, operatively coupled to camera 14, a head-motion analyzer 22 operatively coupled to positional/inertial sensor 16 and a sound and pressure generator 24 operatively coupled to one or more and preferably bilateral ear probes 18. Those of skill in the art will appreciate that, within the spirit and scope of the invention, such operative coupling can be physical or wireless, e.g. telemetric. Nystagmus analyzer 20 outputs nystagmus coordinates and head/torso motion analyzer 22 outputs sway coordinates to what will be referred to herein as an expert subsystem 26. Sound and pressure generator/feedback mechanism 24 is controlled by expert subsystem 26 to generate desirable pressure puffs (sharp, brief attack pressure envelopes) or sounds of given duration, progression, amplitude and frequency to ear probes 18. Ear probes 18 and sound and pressure generator and feedback mechanism 24 will be understood also to generate feedback signals and/or data to expert subsystem 26, as will be described in more detail below be reference to FIG. 3.

One or more of positional/inertial sensor or sensor 16 can be body worn or head worn or both. It is known that a body worn monitor typically is more effective in detecting a sway response to stimulus, since the head's volitional or involuntary tilt might mask a true-positive subject's response or might misrepresent a true-negative subject's non-response as a true-positive response. In accordance with one embodiment of the invention, a single body-worn positional sensor 16 is affixed to the subject's shoulder, as shown in FIG. 2, and effective measures three-dimensionally or omni-directionally, e.g. it is capable of measuring yaw, pitch and roll although only two of these are used in accordance with one embodiment of the invention. Positional sensor 16 can be a global positioning system (GPS) device that indicates absolute three-dimensional position, or it can be a relative positional device that indicates three-dimensional movement. In either case, software within computer 40 that is represented in FIG. 1 as head/torso motion analyzer 22 analyzes position or motion and maps and displays the subject's sway response on display screen 36 if desired.

Many alternatives to the positional sensor of the disclosed embodiment are contemplated, and are within the spirit and scope of the invention. One alternative is an inertial sensor, or accelerometer that detects when the sensor moves from a predetermined position and thus indicates the subject's sway response to stimuli. Another is an inclinometer that detects tilt or incline of the subject out of a baseline angle. Such an inclinometer would be configured to sense spatial orientation rather than position. Still another is an angular accelerometer that detects not only angular movement but also measures such angular movement as a function of time. Thus, any suitable body- or head-worn sensor or other device that is capable of producing data that can be interpreted as indicative of the subject's sway response to stimuli is within the spirit and scope of the invention.

Those of skill in the art will appreciate that, within the spirit and scope of the invention, signal conditioning portions of analyzers 20 and 22 and generator/feedback mechanism 24 can be physically configured within a headset near ear probes 18 or can be housed in one or more separate enclosures external thereto. Also within the spirit and scope of the invention, signal analysis portions of analyzers 20 and 22 can be implemented in software within computer 40, as illustrated. Generator/feedback mechanism 24 can be physically configured on an I/O expansion board within a card slot within expert subsystem 26, described immediately below. Those of skill in the art will appreciate that, preferably within the headset, signal conversion and conditioning from raw to predictable and relatively well-behaved digital form is contemplated, thereby to render as straightforward, accurate, reliable and inexpensive as possible the conveyance via tether (via one or more ‘umbilical’ cords) or telemetry (IR or radio frequency (RF) or other suitable means) of data from the headset to the analyzers and from the generator to/from the ear probes.

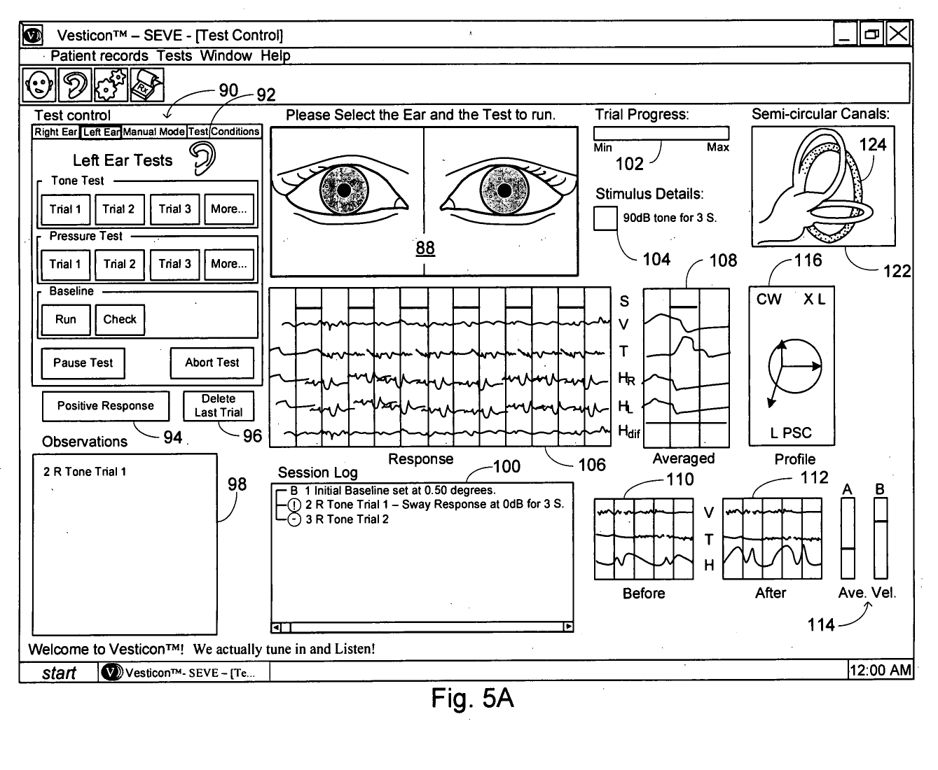

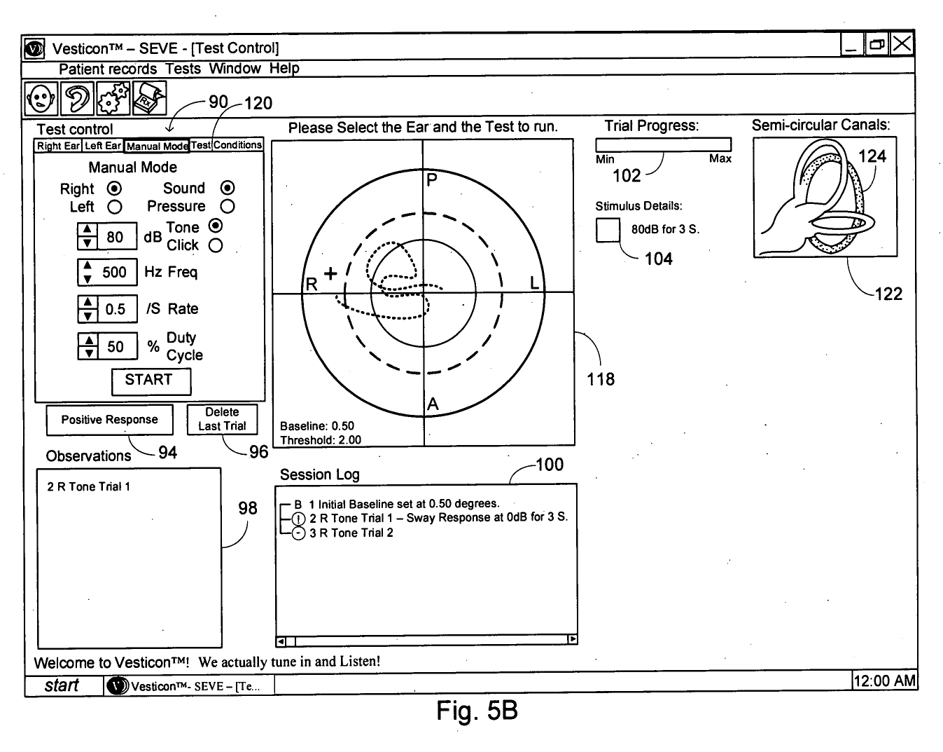

Expert subsystem 26 in accordance with one embodiment of the invention includes a processor 28, a memory 30 for storing instructions and arguments for execution and test parameters/criteria 32 that provide subsystem 26 its so-called ‘expertise.’ Those of skill in the art will appreciate that test parameters/criteria 32 at least semi-automate and thus standardize testing and detecting the nature and extent of a subject's vestibular disorder to assist an operator of system 10 in analysis, detection and even treatment of vestibular condition determined to be pathological. Test parameters/criteria 32 thus are empirically derived from baseline and historic subject observation and treatment, and equip the operator of the system with assistance in distinguishing between normative vestibular conditions and patho-physiological vestibular conditions. Expert subsystem 26 will be described in more detail below by referenced to FIGS. 5A and 5B.

System 10 further includes a keyboard 34 including an associated cursor control device such as a mouse (not shown) to enable inputs to and control of expert subsystem 26 by the operator. System 10 further includes a display screen 36 to enable display and presentation of data, text and graphic representations of stimulus/response or other subject or test condition data to the operator. As indicated in somewhat simplified form in the split screen schematic illustration of FIG. 1, display screen 36 can assist the operator in expertly analyzing ocular and/or sway response to postural and/or sound and/or pressure stimuli. Finally, system 10 includes a destabilization device 38 for intentionally posturally destabilizing a subject standing at least partly thereon, as will be described further below. Those of skill in the art will appreciate that, during a subject test session, different sound and/or pressure test parameters and/or criteria can be used to probe the subject, and different postural conditions, can be controlled by the operator to evoke different ocular or postural sway responses from the subject, in order to detect and perhaps diagnose and even treat a subject determined to suffer a vestibular disorder.

It will be appreciated that the level of actual “diagnostic” use of system 10 is within the discretion of the operator. Thus, the invention contemplates any level of diagnostic use of the invented system, method and apparatus ranging from simply viewing raw or representational subject data on a display screen and drawing mental conclusions therefrom to using the expert subsystem's analysis and display tools conclusively to identify a candidate semicircular canal's otologic cause of a subject's vestibular disorder. Those of skill in the art will appreciate that typical uses of the invented system, method and apparatus will rely on both the analytic “skill” of the expert subsystem and the analytic skill and experience of the operator. In any event, it is believed that detection and treatment of vestibular disorder is facilitated and assisted and standardized to a desired extent, in accordance with the invention.

For example, an operator might use the invention only to assist in determining whether the subject has a vestibular disorder, a determination representing only a first level of analysis. Or an operator might use the invention further to assist in determining which of the subject's ears exhibits a vestibular disorder, a determination representing a second level of analysis. Or the operator might use the invention to assist in determining which canal of which ear of the subject is the likely cause of the vestibular disorder, a determination representing a third and rather advanced level of analysis. At an even more advanced level of analysis, a candidate subject ear canal can be identified on a display device, e.g. graphically or otherwise, as by highlighting. These and even more or less advanced uses of the invented system, method and apparatus to aid in testing, detecting and/or assessing vestibular disorder are contemplated as being within the spirit and scope of the invention.

Those of skill in the art will appreciate that multiple processors can be used to implement any part of system 10, including nystagmus analyzer 20, head/torso motion analyzer 22, sound and pressure generator and feedback mechanism 24 including microcontroller 54, expert subsystem 26 and computer 40. Specifically, multi-tasking and/or parallel or pipeline processing is contemplated by which processing speed advantages are multiplied by running multiple concurrent or simultaneous processors and/or processes and managing concurrent or simultaneous so-called threaded or parallel or pipelined tasks performed by each. It will also be appreciated that processes and processors described herein, within the spirit and scope of the invention, can be implemented in hardware, firmware, software or any suitable combination thereof.

Those of skill in the art will appreciate that determining whether a subject exhibits a vestibular disorder involves a logical sequence of determinations that can be described as a test protocol. First, it must be determined whether there is a measurable response from the pressure/sound stimuli, i.e. whether the subject experienced an ocular or sway response thereto. Then the level of the response must be determined to ascertain whether such is normal or abnormal for the given subject. (Those skilled in the art will appreciate that, at some sound (decibel) level, everyone's vestibular system would be stressed. Thus, it must be determined whether a given threshold sound or pressure level, e.g 40 db, has been exceeded.) Such is often determined by comparing a given response to a historic baseline response recorded when the subject was not under the influence of the stimuli. Then it must be determined in which ear the response was evoked. This so-called ‘localization’ of the vestibular dysfunction is greatly facilitated by the provision of bilateral ear probes that require no intervention to test one subject ear and then another. Further localization can determine which semicircular ear canal is the likely cause of the vestibular dysfunction. Any or some or all of the above localization steps are greatly facilitated and to some extent semi- or fully automated by the present invention.

Those of skill in the art will appreciate that analyzers 20 and 22, expert subsystem 26, keyboard 34 and display 36 within the spirit and scope of the invention can take the form of a desktop or portable laptop computer 40, a personal digital assistant (PDA) or other small, lightweight, portable electronic device having at least data processing capability and preferably also having data displaying capability. Those of skill in the art also will appreciate that body-worn apparatus 12 includes a small, lightweight, portable headset 44 that at least partly integrates cameras 14 and ear probes 18 and one or more head/body sensors 16, as shown in FIG. 2. Indeed, either or both of the headset and sensor(s) can be battery operated. Those of skill in the art also will appreciate that sound and pressure generator 24 can be battery operated and can be separate from computer 40 or integrated therein, e.g. in the form of a custom input/output (I/O) card that plugs into a motherboard or I/O slot. Moreover, those of skill in the art will appreciate that destabilization device 38 broadly refers to any device particularly configured posturally to decrease the baseline stability of (i.e. to destabilize) a subject, e.g. it may take the form of a small step or incline or deformable footpad or cushion (e.g. a loosely fluid- or air-filled bladder, as shown) that, due at least in part to the subject's decreased baseline stability (e.g. balance shift) thereon tends to dis-equilibriate the subject. Thus, preferably device 38 is also small, lightweight and portable.

In accordance with one embodiment of the invention, then, all components of system 10 can be contained for deployment and transport within a portable carrying case 42, indicated schematically in FIG. 1 as a rectangular dashed outline. Case 42 preferably is equipped with a grippable handle. This enables system 10 to be easily transported for field use in even difficult-to-reach locations and conditions where stable power is not readily available.

The right half of the split display screen 36 (to the right as the operator views the display screen and to the right in FIG. 1) illustrates in a top center graphic image an electronic, e.g. videographic, representation of the subject's ocular, e.g. nystagmatic, response to sound and/or pressure stimuli. (Alternative to a videographic representation, those of skill in the art will appreciate that an electronic representation might involve eye muscle positional/motion probes attached to the subject's face to produce two-axis or three-axis coordinate ocular response data. Thus, any suitable 2D or 3D electronic representation of a subject's ocular response to stimuli is contemplated as being within the spirit and scope of the invention.) Such electronic representation will be understood to be captured in memory 30 from cameras 14 via analyzer 20 and to represent a real-time or time-delayed (instant replayed, fast-motion or slow-motion is possible!) representation of the subject's ocular response to stimuli the nature, level and timing of which are also recorded in memory 30 for correlation purposes. Thus, this split screen half shows the subject's left eye and stimulus/response waveforms (traces) and tabulations representing the recently historic stimulus/response for testing and detecting vestibular disorder.

The left half of display screen 36 (as viewed by the operator and as shown in FIG. 1) illustrates a progressive postural sway path experienced by the subject in response to the same or different stimuli, e.g. postural or sound and/or pressure stimuli. The inner circle of a top center graphic image represents the outer limit of a normal sway diameter determined under no stimulus for seven seconds or so nominally to represent a subject's baseline sway, whereas the intermediate dashed circle represents a sway range outside of which a subject's sway response to stimulus is considered to be abnormal and, finally, the outer circle represents a normal fall boundary of approximately ±6.5 degrees from center. The + sign represents the endpoint of the meandering path (so-called ‘mouse tracks’ represented in the drawings by a dashed line) through which the subject's postural sway response progressed before it became potentially dangerous to the subject.

These display screens will be described in more detail below by reference to FIGS. 5A and 5B. (Those of skill in the art will appreciate that stimulus/response data and graphics representing sequential or simultaneous testing of left and right ears are coded on a preferably full color display screen in blue and red, in accordance with convention, but that such color coding is not possible within imposed patent application drawing constraints.)

From the above, it will be understood that the invented SEVE system and method thus features aural stimuli and, optionally, postural conditioning or biasing to test a subject's vestibular function. Postural biasing can include an intentional destabilization of subjects that are nominally stable to evoke a vestibular response. Aural stimuli can include one or both of aural pressure and sound introduced into one or both of the subject's ears.

The invented SEVE system and method also features two different responses including the subject's postural sway response and the subject's ocular response. These different responses are analyzed by the operator of system 10 in conjunction with expert subsystem 26 to make certain determinations regarding the subject's vestibular responses to various stimuli.

Sway response determination. The testing in accordance with one aspect of the invention is carried out in the following manner, although those of skill will appreciate that variations are contemplated as being within the spirit and scope of the invention. The subject stands facing the tester, protected from falling by either a person standing behind them, a hanging harness or a protective corral. They are placed into a condition of minimal stability, ranging from a wide stance on a firm surface with eyes open for a person with severe imbalance, to a condition of feet together or apart above what will be referred to herein as a destabilization device, e.g. an inclined planar surface or a compliant surface such as a foam pad, with one or both feet thereon and with eyes closed, for the person with good postural control. The headset is placed on the subject's head, with air-tight inserts into each ear canal, infrared cameras facing the subject's eyes in infrared light, and one or more position sensors placed on the subject's head and/or body. Then, the test subjects' baseline sway parameter, without stimulus presentation, is determined, documenting the extent of their head/body sway under the given conditions, and displaying this parameter graphically on the computer display screen.

The general technique is to detect and to quantify, if abnormal, the sway threshold level for sound and/or pressure in each ear, and then to present trains of stimuli based upon these levels to detect and analyze any evoked nystagmus. First, sound and/or pressure stimuli are presented, generally to one ear at a time and generally beginning at a low intensity and ascending in a given paradigm, while the subject's postural sway and ocular movements are noted on the screen. Alternatively, sound and/or pressure stimuli are presented to one ear and then the other at progressive levels in accordance with the given paradigm. As noted herein, it is the novel bilateral configuration of the (two) ear probes (or headphones) that renders practical this alternate presentation of sound and/or pressure stimuli first to one ear and then to the other, alternately and repeatedly, without the distracting and time-consuming burden of having to physically re-insert, re-fit and re-seal a single ear probe within one ear and then another and then the first and so on and so on.

If the induced sway under the stimuli demonstrates that they have exceeded certain limits in relation to their baseline, a positive response for that ear and that stimulus is registered, whereupon the stimulus stops so as to limit the induction of nausea, and the final intensity level and grounds for the positive response are documented.

Similar test trials are carried with the same and contra-lateral ear, with both sound and pressure. Different types of stimulus presentation and response analysis are applied, as described below, in a paradigm designed to limit nausea, as well as to counteract, with respect to the sway response, malingering, adaptation and response suppression. During this phase, infrared video of the eye movement is acquired, recorded and displayed.

Alternatively or additionally, bilateral concurrent (overlapping in time) or even simultaneous (starting and stopping at the same time) sound and/or pressure stimuli can be presented to the subject's ears.

Advantages of the bilateral probes (or headphones) and bilateral aural stimulation are that frequent changes in the ear to be stimulated can be undertaken without changing the inserts, and in certain tests the stimuli may be presented to both ears concurrently or even simultaneously. For example, as will be described in more detail below, there is an adaptation response of some subjects to being tested, a naturally compensatory scheme that typically is only subconscious, but that can mask an evoked response to stimuli and thus produce a false-negative vestibular disorder diagnosis. But in accordance with the invention, an adaptation response can be detected and perhaps worked around by the application in the subject's ears of simultaneous sounds one of which intentionally and characteristically masks the other's stimulus. Another occasional test subject response called malingering—by which a subject can produce a false-positive indication of vestibular disorder—also is avoided in accordance with the invention, as will be described below.

Other applications of concurrent or simultaneous application of pressure and/or sound to a subject's bilateral ears are contemplated as being within the spirit and scope of the invention, and are rendered possible by the invented system and methods. Nevertheless, those of skill in the art will appreciate that, even if overlapping (concurrent) testing of a subject's ears is not needed or desired, it is still a great advantage of the invention to provide a headset with (two) bilateral ear probes (or headphones). This is because no change of ear probe configuration is required during a complete testing session and thus no repeated and time-consuming re-seating of an ear probe within the subject's ear canal is needed. Those of skill in the art also will appreciate that providing bilateral ear stimulus devices within the headgear that forms a part of the invented system and apparatus makes testing more repeatable and standardized because the stimulus devices automatically, reliably (and sealingly, in the case of the insertable ear probes 18 described and illustrated herein) seat themselves within the subject's corresponding ear canals once and for the duration of a complete test session.

Ocular Response Determination. Repeatable stimuli and standardized response data representation is provided by the invention using the “Ocular Response” graphical user interface (GUI) represented on the right side of split display screen 36. Thus, the operator manually or the expert subsystem automatically selects the recommended parameters for presentation of a “train” of the stimulus that is selected, whether sound or pressure or both. By testing the subject concurrently for ocular response and for sway response, an operator can learn a great deal in a short time about a subject's vestibular condition. Those of skill in the art will appreciate that the principle ocular response indicative of vestibular disorder is nystagmus. Thus invented system 10 focuses on collecting, analyzing or converting and presenting data related to nystagmus that might be detected in a subject's ocular response to air pressure and/or sound stimuli. Other responses also are detectable, using the invented system and method, however, including binaural ocular convergence, which recently has been deemed a possible indicator of vestibular disorder. Within the spirit and scope of the invention, any and all ocular responses are contemplated as being detected by use of the invented system and method.

Any suitable test protocol can be used, including traditional Colebatch-scripted testing or any suitable alternative.

The ocular response of one ear is viewed in display screen and tracings of its 3-D components (horizontal (H), vertical (V) and torsional (T)) are displayed relative to each stimulus, and then averaged. The likely semicircular canal of origin (Profile) is determined from vector analysis. Optionally, the candidate semicircular canal that is the most likely cause of the vestibular dysfunction is identified and highlighted in graphic form, e.g. in area 122 (indicated schematically in FIGS. 5A and 5B by stippling). Binaural horizontal components (right horizontal (H_{R}) and left horizontal (H_{L})) are compared (H_{dif}) to demonstrate any ocular convergence response, which can be an indicator of vestibular dysfunction. To determine the production of increased ocular activity by a stimulus, segments before and after the “stimulus run” are displayed and the average velocities (of all components) are compared.

The eye video and 3-D components of the evoked ocular responses are displayed graphically in the time domain in relation to the stimuli. The evoked responses are averaged in order to differentiate them from artifact, after extraneous artifacts are rejected. Vector analysis of these averaged responses is used to identify the potential semicircular canal(s) of origin. The ocular convergence response is detected by comparing the horizontal component of one eye with the corresponding component in the contra-lateral eye as the stimulus train evokes ocular responses. These are also averaged. Those of skill in the art will appreciate that, within the spirit and scope of the invention, ocular response analysis can proceed in an alternative manner to that described and illustrated herein.

SEVE system 10 is designed to provide an efficient way to identify and assess these sound and pressure evoked phenomena by at least semi-automating and perhaps even fully automating the use of sound and/or pressure stimulus and/or response tests to assess a subject's adverse responses thereto that might indicate vestibular dysfunction or disorder or pathology. The computer-based system, method and apparatus of the invention can be understood to facilitate both qualitative (e.g. Does the subject have a nystagmatic or sway response to the sound and/or pressure stimuli?) and quantitative (e.g. Is the subject's nystagmatic or sway response to the sound and/or pressure stimuli pathological?) subject evaluation and assessment. Such information gleaned from the invention can be used by an operator in a clinical or field setting for ongoing diagnosis and treatment of the subject.

Briefly summarizing, system 10 utilizes sound and pressure evoked phenomena to assess both the subject's vestibulo-ocular (nystagmus) and vestibulo-spinal reflex (postural) systems. Importantly, such ocular and sway responses by a subject can be simultaneously observed by the use of system 10. System 10 includes body-worn apparatus 12 that includes a set of video eye goggles to conduct 3-D vector analysis and quantification of patho-physiological nystagmus, and also consists of spatial orientation and motion sensors (inertial sensors 16) to quantify postural (head/body) instability. The apparatus further includes a computer system for stimulus control, response data acquisition, analysis, display and documentation. In operation, the apparatus generates variable sets of sound (tones, clicks) and/or air pressure (positive and negative) stimuli in one or the other ear and will simultaneously record, interpret and display the stimulus-response data from the goggles and inertial sensors on a display screen 36. An optional printed report and a digital tape recording of the screening results would provide documentation for reimbursement and medico-legal purposes.

FIG. 2 is an isometric and somewhat schematic view of invented system 10 including a headset 44 mounted on the head of a subject, inertial sensors 16 mounted on the torso, e.g. a shoulder, of the subject and computer 40 operatively connected thereto. Those of skill in the art will appreciate that besides cameras 14, headset 44 includes a generally U-shaped, laterally symmetric and curved body 46 that includes a live spring portion 48 made, for example, of spring steel, that gently arcuately biases bilateral (left and right) ear probes 18a and 18b inwardly in the direction of the inwardly, upwardly arched opposing arrows to auto-insert themselves within the subject's ear canals in operative generally axial alignment therewith and with the outer generally cylindrical periphery of each of the left and right ear probes 18a and 18b engaging the inner wall of the corresponding left and right ear canal.

It will be appreciated that, when the lateral ear-insert halves of headset 44 are biased apart slightly, by hand, as by the operator or the subject, bilateral ear probes 18a and 18b easily laterally encompass the face and ears of the subject and are slightly wider apart than are the subject's ears. Conversely, when the lateral ear-insert portions of headset 44 are permitted gently to return toward their spring-loaded or biased position nearer one another, they instead auto-insert themselves in the bilateral ears of the subject in such manner that they sealingly engage the same in an operative alignment. Importantly, there is enough shape-retentiveness or so-called ‘memory’ in the headset spring structure generally to maintain the overall U-shape of the structure and to maintain the overall inwardly opposing alignment of the ear probes but enough flex therein to yield and deform slightly so that individual head shapes, sized, ear locations sizes and ear canal idiosyncrasies are accommodated while the spring urges the probes into proper operational and sealing alignment within the subject's ear canals.

Those of skill in the art will appreciate that ear probes 18 include air conduits and an umbilical cord feeding sound and/or pressure stimuli from sound and pressure generator 24 under control of expert subsystem 26 operating within desk/laptop computer 40. It will be appreciated that computer 40 is shown in FIG. 2 as a laptop computer, but that computer 40 can take the alternative form of a PDA or other handheld device having a soft or hard keyboard 34 and a display screen 36. It will also be appreciated that generator 24 can take the form of a small lightweight battery operated or USB operated portable device. In any event, those of skill in the art will appreciate that lightweight portability is possible regardless of the form computer 40 takes, as carrying case 42 can take the form of a briefcase or computer tote bag facilitating the easy lifting or wheeling and thus transporting of system 10.

Thus, while clinical and relatively fixed-location use of system 10 is possible and contemplated as perhaps a more typical environment, it is a great advantage of the invention that system 10 is portable and lightweight and can be battery operated and thus transported in the field to relatively remote sites for subject testing. For example, field testing of troops disposed in mobile combat is possible, or field testing of civilians disposed in remote and rugged terrains is possible. Indeed, it is the small, lightweight, portable all-in-one nature of the invented system that lends it so well to vestibular disorder testing and to sway and nystagmus response data gathering and recording and perhaps also vestibular disorder detection, diagnosis and treatment.

Those of skill in the art will appreciate that computer 40 can be straightforwardly equipped with a wired or wireless connection with a local area network (LAN) or a wide area network (WAN) such as the Internet, as suggested by the comm-link bolt in FIG. 2. With appropriate encryption, as needed, to satisfy patient data privacy concerns and/or government regulations (e.g. the patient data privacy parts of Hospital Insurance Portability and Accountability Act (HIPAA), data integrity and privacy can be assured, while computational flexibility and archival capability can be greatly increased. Alternatively, but within the spirit and scope of the invention, subject data can be stored in local memory 30 and can be used to generate written tables, graphs and reports for comprehensive long-term patient treatment or medico-legal archival purposes. Those of skill in the art will appreciate that memory 30 can be a fixed permanent store such as a hard disc drive or a temporary store such as a volatile read-and-write memory (RAM). Memory 30 alternatively can be a removable/transportable memory such as a portable, non-volatile memory stick, flash memory, CDROM or diskette.

Those of skill in the art will appreciate that, within the spirit and scope of the invention, other input/output (I/O) devices such as modems, readers, printers or facsimile machines can be used with the invented system. Thus, medical practice advances such as telemedicine are contemplated as broadly including one or more of remote testing, analysis, presentation, diagnosis and even prescriptive medication are possible in accordance with bi-directional audiovisual (AV) telecommunication protocols that utilize existing wired or wireless infrastructure.

Preferably bilateral digital cameras 14 in accordance with one embodiment of the invention are housed in a pair of goggles 50 that fit around the subject's head and over the subject's ears, as illustrated in FIG. 2. Goggles 50 will be understood generally sealingly to engage the subject's face such that little ambient light is allowed to enter the lenses of digital cameras 14. Those of skill in the art will appreciate that digital cameras 14 produce digital IR video images that, in accordance with one embodiment of the invention, are conveyed via a so-called Firewire port (IEEE 1394). Those of skill in the art also will appreciate that, within the spirit and scope of the invention, one or more digital video images (representing the subject's left and/or right eyes) can be conveyed to computer 40 via any suitable interface and into any suitable port, e.g. universal serial bus (USB) and in any suitable form and in accordance with any suitable protocol. No digital signal processor (DSP) is used in accordance with one preferred embodiment of the invention, although such pre-processing is certainly within the spirit and scope of the invention. Instead, in accordance with one embodiment of the invention, digital video data and other data, e.g. inertial sensor data, analysis is performed by application software executing in expert subsystem 26 within computer 40.

Alternative video recording and presentation means are contemplated. For example, one or more analog cameras can be used to produce an analog representation of the subject's eye movement, the analog signals being converted by suitable means, e.g. an analog-to-digital converter (ADC), to digital form for recording in memory and for presenting on display screen 36. Thus, any suitable videographic or other electronic ocular response recording and display means are within the spirit and scope of the invention.

Destabilization device 38 will be understood by those of skill in the art to take any suitable form including the simple malleable but somewhat shape-retentive cushion form illustrated in FIG. 2. Some subjects are difficult to test for vestibular disorder because they exhibit normally relatively stable posture. These subjects can be destabilized to better test their vestibular conditions. One simple way to decrease such a subject's baseline stability is to cause his or her equilibrium or sense of vertical balance to be challenged by having the subject stand on a malleable cushion. Those of skill in the art will appreciate that destabilization device 38 alternatively can take the form of a tiltable platform having a central pivotal fulcrum on which the subject stands, the platform either being passive such that the subject must tilt it or active such that a motor drive tilts it. In any event, the platform tilts and the subject reacts to the tilt because the subject is thrown into disequilibrium or relative baseline instability.

The extent of the destabilization experienced by the subject under the influence of destabilization device 38 is relatively unimportant, so long as the subject's baseline stability is challenged, i.e. decreased. Nonetheless, if desired, the extent can be controlled and/or measured by rendering a destabilization platform in the form of a force plate having peripheral pressure transducers that tilt the pivotal plate out of horizontal and having sensors that measure the extent that the pivotal plate is out of horizontal. One such complex, heavy and expensive body destabilizer is described in U.S. Pat. No. 4,738,269 to Nashner et al. issued Apr. 19, 1988. Such apparatus as is described therein can be used in conjunction with the invented SEVE system intentionally to destabilize a subject for vestibular function testing, within the spirit and scope of the present invention.

But one great advantage of using a simple, passive destabilization device 38 such as a two-footed compliant pad or cushion that forces the subject into baseline instability is the small, lightweight, passive device's portability. No heavy or complex platform or electronics or power access is required and thus the invented system can be used outside a clinical setting in a field setting. This is because of the lightweight portability and deployability of the invented system. In accordance with one embodiment of the invention, mobile troops, for example, or remote or nomadic populations, for example, could be tested easily and cost-effectively for vestibular disorders. Thus, all important components of system 10 including destabilization device 38 fit neatly within portable carrying case 42.

Those of skill in the art will appreciate that standardized and semi-automated testing and recording of clinical or field trials of subject stimuli/response represents an important advantage over prior art, ad hoc testing and reporting. Thus, system 10 facilitates subject vestibular condition testing and vestibular disorder detection, diagnosis and treatment. Raw or processed data can be recorded for later oversight and analysis. More important, perhaps, processed data can be presented in representational form on a display screen integral with the system for instant analysis and detection by the operator or the machine (e.g. the computer), and for possible treatment. With the expert subsystem operating within computer 40, an operator can stimulate a subject in a measured and predetermined way and can measure and evaluate the subject's nystagmatic and/or sway response thereto also in a measured and predetermined way. Thus, testing, detection and treatment are semi-automated and greatly facilitated. A permanent record can be made for archival purposes.

FIG. 3 is a detailed schematic diagram of sound and pressure generator and feedback mechanism 24. Generator/feedback mechanism 24 includes a computer interface 52 and microcontroller (μCONTROLLER) 54 operatively coupled with one another. Computer interface 52 will be understood to be operatively coupled with computer 40. Those of skill will appreciate that microcontroller 54 is programmed to provide control and monitoring interconnections with the remaining functional blocks to be described below, although for the sake of clarity no such operative interconnections are shown. For example, microcontroller 54 can implement a state machine or other flow control mechanism that is hardwired, firmware-controlled (e.g. as by use of a read-only memory (ROM) or programmable logic array (PLA) or the like or software-controlled, e.g. using a writeable control store (WCS). Microcontroller 54 can be as simple as a look-up table- or ROM-based controller that enforces operational parameters for the sound and pressure and feedback mechanisms. Those of skill in the art also will appreciate that there are two nearly identical circuits for the left ear probe 18a and for the right ear probe 18b indicated by identical reference designators with a corresponding “a” or “b” suffix.

Thus, a left channel sound and pressure generator and feedback mechanism includes stimulus/tone generator 56a, precision gain/attenuation device 58a and power amp 60a producing programmed sound levels, tonal qualities and dynamics; air system 62a and acoustic filtering device 64a producing programmed pressure levels and dynamics; and amplifier 66a and analog-to-digital (A-D) converter (ADC) 68a producing a feedback signal representing sound and/or pressure and other conditions corresponding to the subject's left ear. Those of skill in the art will appreciate corresponding functional blocks 56b, 58b, 60b, 62b, 64b, 66b and 68b identically produce programmed pressure levels and dynamics, programmed sound levels, tonal qualities and dynamics and a feedback signal representing sound and/or pressure and other conditions corresponding to the subject's right ear. Stimulus/tone generator 56a, precision gain/attenuation device 58a and power amp 60a will be referred to herein collectively as a sound generator. Air system 62a and acoustic filtering device 64a will be referred to herein collectively as an air pressure generator.

Those of skill in the art will appreciate that, for the sake of clarity and brevity, left and right ear probes 18a and 18b are identical and thus only left ear probe 18a will be described in detail below.

Left ear probe 18a includes a tapered, generally cylindrical earpiece 70 containing an air tube 72, a miniature speaker (referred to in the audiology literature as a “receiver”) 74 and an optional microphone 76. Air tube 72 is securely connected to an air hose 78 via an irregularly shaped, interference fitted connector 80. The distal end of air tube 72 extends to a hollow distal region 82 of earpiece 70, as shown. The proximal end of air tube 72 is in fluid (air) communication via air hose 78 with acoustic filtering device 64a/b. Speaker 74 and microphone 76 are respectively connected to power amp 60a/b and amplifier 66a/b via a wiring harness 84 containing at least two signal wire pairs, e.g. twisted signal wire pairs, as shown (although those of skill in the art will appreciate that some microphones provide a third bias terminal and thus require three wires instead of two). The distal ends or so-called ‘snouts’ of speaker 74 and microphone 76 also extend to hollow distal region 82 of earpiece 70, as shown.

In accordance with one embodiment of the invention, air tube 72, speaker 74 and microphone 76 are secured within earpiece 70 by potting compound 86, e.g. of epoxy resin. In accordance with one embodiment of the invention, potting compound 86 extends only partway toward the distal end of earpiece 70, thus forming hollow (air-filled) distal region 82 that is in fluid (air) communication with the subject's left ear. Alternatively, yet within the spirit and scope of the invention, the potting compound can be of any suitable material that instead may fill the entire interior region of earpiece 70. In accordance with one embodiment of the invention, earpiece 70 is of machined or extruded aluminum, although within the spirit and scope of the invention it can be of any suitable material. Those of skill in the art will appreciate that earpiece 70 is configured to have an ear-canal-easy cushion, not shown, slipped over its distal end for the comfort of a test subject.

A described above, headphones can be used instead of ear probes 18 to deliver sound (but not pressure) to the subject's ears. Those of skill will appreciate that such a configuration of system 10 would require only parts (i.e. those including the sound generator circuitry of sound/pressure generator/feedback mechanism 24 to be active during subject testing. Such an alternative configuration of ear stimulus equipment nevertheless permits sound stimulation of a subject and ocular and/or sway response that might indicate vestibular function anomaly or pathology. This and other alternatives to the use of insertable and sealingly engageable ear probes 18 for sound and/or pressure testing all are contemplated as being within the spirit and scope of the invention.

Those of skill in the art will appreciate that the two-channel sound and pressure generators and speakers 74 are used in accordance with the invention to deliver sound and/or pressure stimuli to the subject's ears. They also can be used, within the spirit and scope of the invention, to deliver voice commands to the subject, in manual test mode, via live spoken words or phrases from an operator or, in automatic test mode, via digitized and recorded (and perhaps real-time synthesized speech) words or phrases from the expert subsystem part of the computer. Those of skill in the art also will appreciate that, within the spirit and scope of the invention, microphone 76, amplifier 66a/b and ADC 68a/b can be omitted entirely from mechanism 24, while still permitting the sound and pressure generator portion of mechanism 24 to remain fully operational but without the benefit of feedback and monitoring. Such alternatives are contemplated as being within the spirit and scope of the invention.

In brief summary, independent control of dual, contralateral, left and right ear probes 18a and 18b is facilitated by the provision within sound and pressure generator and feedback mechanism 24 of separate left and right channel controls described above. Thus, by programming computer 40 and microcontroller 54 appropriately, a subject's left and right ear can be stimulated one at a time, concurrently or simultaneously, with identical, similar or completely different stimuli, e.g. preferably substantially pure sine waves or so-called ‘clicks’ in the case of sound or puff envelope stream. This is of great benefit in testing subjects for vestibular disorder, since subjects can have very different left and right hearing and balance capabilities and since a variety of stimuli typically might be required to produce a desired (revelatory) response. By providing sound and/or pressure stimuli in accordance with empirically derived protocols, subject testing can be semi-automated and greatly facilitated. Those of skill in the art will appreciate that computer 40 including test parameters/criteria 32 in accordance with the invention straightforwardly is programmed to interface generator/feedback mechanism 24 in such manner that helpful stimuli are produced and responses thereto recorded and analyzed, as will be described in some detail below. A script of a typical sound and/or pressure test session is discussed below by reference to FIGS. 5A and 5B.

Those skilled in the art will appreciate that, depending upon the level of intelligence or expertness that is provided in accordance with a particular embodiment of the invention, a test subject can also be an operator of the system or apparatus. In other words, a high-level of integrated intelligence within a system or apparatus made in accordance with the teachings of the present invention could enable a portable system 10 so easy to use and automatic that it could be used by a subject, who would become the operator for purposes of beginning canned test sequences, observing and recording test results and, perhaps uploading such vestibular function test results to a remote data site for oversight by a qualified vestibular function specialist. Such remote and essentially unsupervised subject testing is greatly facilitated by the invention, which can use data conveyances technologies and infrastructures effectively to realize what might be referred to as supervised self-testing, which might be thought of as the testing phase of telemedicine.

Those of skill in the art will appreciate that microphone 76, amplifier 66a/b and ADC 68a/b that form part of the feedback mechanism are used in accordance with one embodiment of the invention to ensure proper sealing of left and right ear probes. 18a and 18b. This is accomplished, for example, by applying a known sound and volume and measuring the acoustic response within the subject's ear as picked up by the microphone. A straightforward comparison of input and output sound parameters determine whether the ear probe is properly sealed and further testing is avoided if the seal is not secure. The display produces a warning to the operator. Thus a first use for this circuitry is during placement of the headset on the subject and during testing of the subject to ensure initial and continuously sealed acoustic engagement with the subject's ear canals.

Those of skill in the art will appreciate however that other uses can be had of the microphone and associated circuitry. For example, tympanometric measures can be made within the subject's ear canals to evaluate middle ear function (acoustic admittance). The phenomenon that makes this possible is the tympanic membrane's (eardrum's) critical position between the outer and middle ear. When there is approximate pressure equilibrium therebetween, the tympanic membrane is flaccid and responds in a different way to sound and pressure stimuli thereon than when there is disequilibrium therebetween suggesting a middle ear canal problem in the subject. (When there is disequilibrium, the tympanic membrane is relatively rigidly configured in a concave or complex shape in response to the differential pressure between outer and middle ear.) Thus, useful testing includes sound and/or pressure tests that determine the level of correlation between a stimulus and a response as respectively produced by and fed back to sound and pressure generator/feedback mechanism 24. Such correlation and other useful analysis and reporting are straightforwardly accomplished by suitable “expert” programming of expert subsystem 26 as part of computer 40 including display screen 36.

Those of skill in the art will appreciate that, within the spirit and scope of the invention, an operator or expert subsystem 26 can base sound pressure level of the test stimulus on the predetermined hearing threshold of the subject or another suitable metric. Such a metric can be measured or not, e.g. it can be empirically or statistically or otherwise determined or derived, within the spirit and scope of the invention. Thus, any suitable metric, whether measured or not, can be used in any suitable manner to determine a suitable test protocol for a given subject.

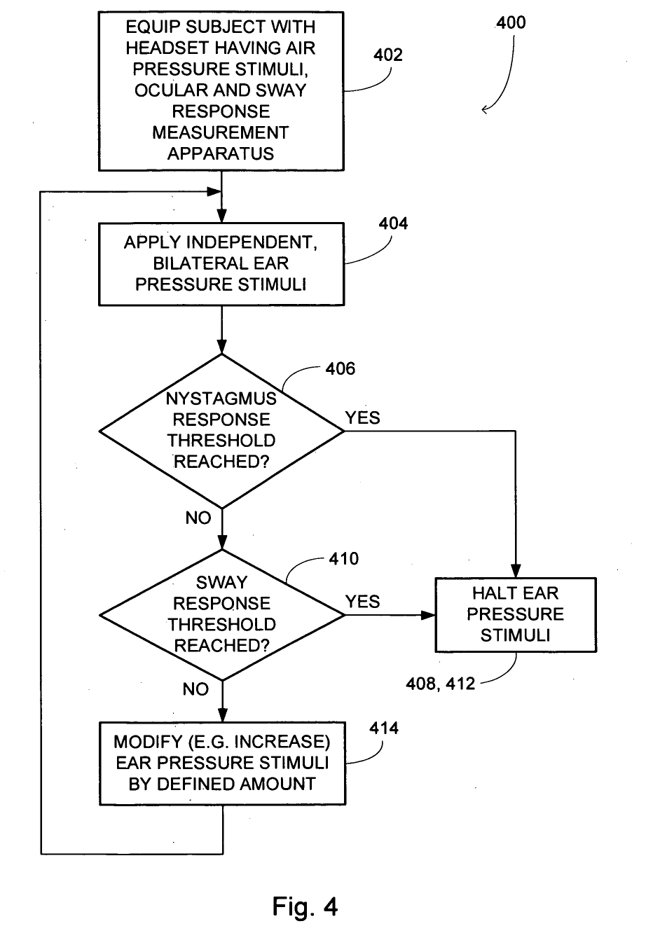

FIG. 4 is a flowchart illustrating the invented method 400 of testing a subject for response to ear pressure stimuli for vestibular condition detection purposes. The method includes a) at 402 equipping a subject with a headset including bilateral intra-ear-canal air pressure stimulus apparatus and b) at 404 concurrently applying bilateral ear pressure stimuli to one ear and to the contra-lateral ear.

(Those of skill in the art will appreciate that the air pressure stimuli can be one or more sound pressure levels (SPLs, as representing subject-recognizable sounds) or the air pressure stimuli can be one or more air pressures as representing sharp-attack pressure envelopes or so-called ‘puffs’ or ‘clicks.’ In other words, air pressure refers to herein as one or more pressures at a frequency that is below the human audible tone, e.g. less than 1 Hz, whereas sound pressure level (SPL) refers to herein as sound recognizable by human subjects as such and being characterized by a frequency within the human audible tone or white noise zone, e.g. between approximately 20 Hz and 8 kHz or perhaps even twice the upper limit (so-called ‘high-audible’). Importantly, the present invention contemplates and enables the delivery to one or both ears of one or both of atonal, i.e. air (e.g. puff or click) pressure stimuli and tonal, i.e. sound pressure level (SPL) stimuli and the assessment of the subject's evoked response to either or both.)

When the headset includes bilateral ocular measurement apparatus, the method further includes c) at 406 detecting a defined threshold level of ocular response, e.g. nystagmus response of the subject to the pressure stimuli. Optionally, upon such detection, the method further includes d) at 408 automatically halting the application of ear pressure stimuli. When the headset includes sway response measurement apparatus, the method further includes e) at 410 detecting a defined threshold level of sway response of the subject to the pressure stimuli. Optionally, upon such detection, the method further includes f) at 412 automatically halting the application of ear pressure stimuli. If the sway response threshold has not yet been reached, then g) at 414 the ear pressure stimuli are modified in a defined way, e.g. increased a predefined incremental amount, and control is returned once again to block 404 for further testing based upon application of modified ear pressure stimuli.

Those of skill in the art will appreciate that expert subsystem 26 and/or test parameters/criteria 32 might at block 414 leave the ear pressure stimuli the same or even decrease the ear pressure stimuli. Thus, the invented method contemplates any intentional modification to the stimuli and reapplication thereof that is suitable in testing for and possibly detecting a vestibular disorder in a subject, in accordance with logic and experience. Those of skill in the art will also appreciate that, before a reapplication of ear pressure stimuli, the modified stimuli can be tested against threshold or subject stress limits before its application to the subject in order to protect the subject against even a momentarily stressful or uncomfortable experience.

FIGS. 5A and 5B represent the left and right parts of the split display screen 36 of FIG. 1, and illustrate the important graphical user interface (GUI) features of the invention as well as the test parameters/criteria 32 used in at least semi-automating and standardizing vestibular disorder testing and detection. Those of skill in the art will appreciate that at least one of sound and pressure is introduced into one or both ears of the subject in accordance with a semi-automated protocol. The protocol preferably is stored in test parameters/criteria 32 of expert subsystem 26 that forms a part of computer 40. Prompts guiding the operator through the application of sound and/or pressure can be presented to the operator of system 10 on display screen 36 of computer 40. The operator then observes the subject and/or representations thereof on the display screen to determine the subject's ocular and/or postural sway response to the sound and/or pressure stimuli. These prompts and test parameters will be described below briefly in relationship to the ocular and sway responses observed in accordance with respective FIGS. 5A and 5B to be described in detail below.

FIG. 5A shows the ocular response GUI or display screen and illustrates how expert subsystem 26 assists an operator in rendering detection, diagnosis and treatment decisions regarding a subject being tested for vestibular function. Those of skill in the art will appreciate that the GUI provides a great deal of functionality in the form of graphics, raw test parameters and raw and interpreted test data Pull-down menus and/or toolbar pushbuttons permit the operator to select various subject data entry and display screens, as suggested. Those of skill in the art will appreciate that expert subsystem 26 and/or computer 40 straightforwardly can be programmed to implement the functions behind such menus and toolbars and to generate suitable display screens and operational behaviors that provide a broad array of desired data entry and display functions.

Because it is believed to be largely self-explanatory, ocular response GUI will be described in detail only in pertinent part. It includes a realistic depiction 88 of the subject's eyes as ‘seen’ by digital IR bilateral eye video cameras 14 and recorded electronically (e.g. videographically) in memory 30. It includes a set of Test control tabs 90 including Right Ear, Left Ear, Manual Mode and Test Conditions tabs. It includes a test panel selected by selecting the Left Ear tab, test panel having an array 92 of pushbuttons for choosing Tone v. Pressure tests in Baseline and numbered Trial runs, while also providing Pause and Abort Test options. It includes separate pushbuttons 94 and 96 for respectively noting a Positive Response and for Deleting Last Trial. It includes an Observations panel 98 for manually recording notes, e.g. using keyboard 34. The ocular GUI also includes a Session Log 100 that automatically records notes about the phases of each test session.

Those of skill in the art will appreciate that test parameters and protocols can be biometrically based, i.e. they may be based upon a real or hypothetical subject's biometrics, whether measured, calculated, predicted or otherwise determined. Thus, whether using the ocular response GUI of FIG. 5A or the sway response GUI of FIG. 5B, the sound and pressure stimuli intended to evoke at least one of an ocular and a sway response in the response are output and one or more test suites proceed under manual (live operator) or machine (programmed) control. The operator using system 10 is able to monitor the progress of the tests by use of either the ocular response GUI of the sway response GUI, as will be described below.

Ocular response GUI shown in FIG. 5A further features a gauge 102 that marks Trial Progress and an icon 104 that summarizes sound and/or pressure Stimulus Details. It includes a set 106 of Response waveforms or traces over plural, e.g. thirteen, successive intervals, as well as a set 108 of Averaged waveforms, for a number of variables including stimulus (S), vertical (V), torsional (T), horizontal right (HR), horizontal left (HL) and horizontal differential (Hdif). It includes a set 110 of averaged Before and a set 112 of averaged After stimulus response waveforms for a number of variables including vertical (V), torsional (T) and horizontal (H). It includes a set 114 of bar graphs indicating the average velocity of the subject's horizontal nystagmus After (A) and Before (B) the stimulus. In accordance with the invention, display 36 as a part of system 10 combines a visual representation of one or more of a subject's aural sound and/or pressure stimulus or stimuli and/or a visual representation of the subject's ocular and/or sway response thereto. The subject response representation preferably includes one or more of the following: a) waveforms or traces of the subject's eye movements in at least two dimensions and averages over time thereof, b) depictions of the subject's sway movements thereto in at least two dimensions and an indication of the test parameters that evoked any such subject response. In accordance with one embodiment of the invention, a display forming a part of system 10 combines a visual representation of both, as suggested by split screen 36 of FIG. 1 and as illustrated by FIGS. 5A and 5B.

Finally, the ocular response GUI or display screen illustrated in FIG. 5A includes a Profile 116 indicating in a three-orthogonal-axes coordinate system the quality of the subject's nystagmus in terms of clockwise (CW), axis left (XL) and left posterior semicircular canal (L PSC). Clearly, from all indications from FIG. 5A for the subject under test, the left posterior semicircular canal is indicated as being the likely culprit in the subject's vestibular disorder, based upon the extent of its vector well outside a circle representing the norm.

It has been discovered that, with some subjects, there is a stimulus/response phenomenon that will be referred to herein as ocular convergence. Such can be detected by use of the ocular response GUI shown in FIG. 5A, especially by contrasting the H_{R} and H_{L} waveforms or traces within the Response region 106 or by analyzing the H_{dif} trace and looking for contralateral, inward movements between the left and right eyes in response to sound and/or pressure stimuli. It will be noted that in FIG. 5A, there appears to be no ocular convergence phenomenon, but the left eye's response in general time-lags that of the right eye. Those of skill in the art will appreciate that equipping headset 44 with dual, bilateral cameras 14, it is possible with system 10 including expert subsystem 26 to display on display screen 36 raw and/or representational data that illustrates an ocular convergence phenomenon that may indicate vestibular dysfunction.

FIG. 5B shows the sway response GUI and illustrates the sway coordinates (data) analysis and graphic presentation features of the invented system. Those of skill in the art will appreciate that, at the highest level, the GUI of FIG. 5B can take the same form as that described above by reference to FIG. 5A. For example, pull-down menus, buttons, Test control tabs (90), Ear Tests pushbutton panel (92), Positive Response pushbutton (94), Delete Last Trial pushbutton (96), Observations panel (98), Session Log (100), Trial Progress gauge (102) and Stimulus Details icon (104) operate and appear identically to minimize GUI complexity and learning curve for the operator. Nevertheless, within the spirit and scope of the invention, the user interfaces can differ from one another and can take alternative forms.

Sway is observed in a panel 118 that depicts an outer solid circle representing an upper sway threshold, a dashed intermediate circle representing a stimulus-evoked intermediate sway threshold and a solid inner circle representing a baseline-established (stimulus-free) lower sway threshold. The so-called ‘mouse tracks’ or irregular dotted line terminated by a + sign represent the subject's meandering sway path that is sound and/or pressure stimulus-evoked, as determined for example by expert subsystem 26 in response to one or more positional/inertial sensors 16. The four labeled axes emanating perpendicularly from the center of the concentric circles within panel 118 represent the right (R), left (L), anterior or front (A) and posterior or back (P) postural sway deviations from the center. Thus, those of skill will appreciate that the invented system, method and apparatus annunciate and depict direction-specific sway response of a subject to stimuli. The Manual Mode tab allows one stimulus at one level to be presented to the subject, whether tone or pressure, i.e. it brings up panel 120 shown for the sake of clarity only in FIG. 5B.

The sway response GUI shown in FIG. 5B features a selection means, via the Manual Mode tab of entry region 120, for manually setting sound and/or pressure test parameters. Those of skill in the art will appreciate that sway response can be tested instead in accordance with predefined test protocols for the Right Ear or Left Ear by selection instead of those tabs in Test control panel 90, as described above for the ocular response GUI of FIG. 5A. FIGS. 5A and 5B are different in this respect to illustrate the versatility and ease of use of the invented system having common, easy-to-use user interfaces for producing ear sound and/or pressure stimuli and for testing ocular and sway response thereto.

Typically, the screen represented by FIG. 5B is designed to assist the operator in recording observations, test protocols, evoked response parameters and data stored in memory and optionally presented in raw or representational form on the computer's display screen. The remaining features of the screens represented by FIGS. 5A and 5B are illustrated for the sake of completeness, can be presented in any suitable configuration and can present any suitable contents, and form no part of this invention.

Those of skill in the art will appreciate that analysis of subject nystagmus response data and identification based thereon of a candidate semicircular canal as the cause of a manifest vestibular disorder proceeds in accordance with the teachings of the above-incorporated patent application. Those of skill in the art also will appreciate that such can proceed cooperatively between expert subsystem 26 and the operator or by expert subsystem 26 alone. In the latter case, it is contemplated as being within the spirit and scope of the invention to provide an indication of the candidate semicircular canal by color, intensity, flashing, shading, cross-hatching or other visible highlighting the same on a 3D facsimile depiction of the candidate canal to distinguish it among the three semicircular canals lying within their three generally orthogonal axes.

Such is shown in FIGS. 5A and 5B at 122, which is a pictorial representation of the semicircular canals anatomy of the subject. Importantly, the depiction at 122 highlights the candidate or suspect semicircular canal 124 of the subject that is the likely cause of the subject's vestibular disorder, as determined by expert subsystem 26 or at least from assistance thereby. Those of skill in the art will appreciate that the visual highlighting of the candidate ear canal exhibiting a vestibular disorder based upon the subject's postural or ocular response to the sound or aural pressure stimulus can take any suitable form, within the spirit and scope of the invention. (Due to patent drawing constraints, it takes the form in FIGS. 5A and 5B only of a stippled semicircular canal 124 (the one that lies generally in a sagittal plane parallel with the subject's median plane) in FIGS. 5A and 5B that readily is visually distinguishable from the two others not similarly indicated by the stimulus-response data. Those of skill in the art will appreciate that this indication is consistent with the graphic Profile 116 indication in FIG. 5A that the subject's ocular response to the sound and/or pressure stimuli point to the left posterior semicircular canal (L PSC)).

In accordance with one embodiment of the invention, a candidate one of six semicircular canals of a given subject is highlighted on display screen 36 by operation of expert subsystem 26. This is done in accordance with one embodiment of the invention by determining, based upon either or both of the subject's nystagmus or sway responses, and based upon anatomy and orientation of the subject at particular times during the stimulus/response trials, which canal is most likely the source of the subject's vestibular disorder. Those of skill in the art will appreciate that the three canals, which are nominally in approximate orthogonal relational orientation to one another, can be straightforwardly depicted graphically in three dimensions on display screen 36. The canal most likely to be causing the subject's disorder is then very simply highlighted as by modulating its intensity or changing its color relative to the rest of the graphic. In this way, expert subsystem 26 presents instruction or guidance data to the operator in any useful form ranging from raw tabulated data to highly processed, analyzed and interpreted data.

Those of skill in the art will appreciate that the invented system, method and apparatus enable the semi-automatic or fully automatic modification of stimuli in nearly or fully real-time response to observed subject ocular or sway response to previous stimuli. For example, if a subject responds to first sound and/or pressure stimuli in a particular way that is understood by expert subsystem 26, then expert subsystem 26 can proceed automatically to alter the test protocol including one or more test variables to further stimulate a response in the subject that more definitively implicates the subject's vestibular disorder. Thus, a defined progression of tests proceed automatically or at least semi-automatically based upon measured or observed and display-represented subject responses to earlier stimuli as system 10 effectively ‘learns’ the subject's idiosyncratic vestibular function.

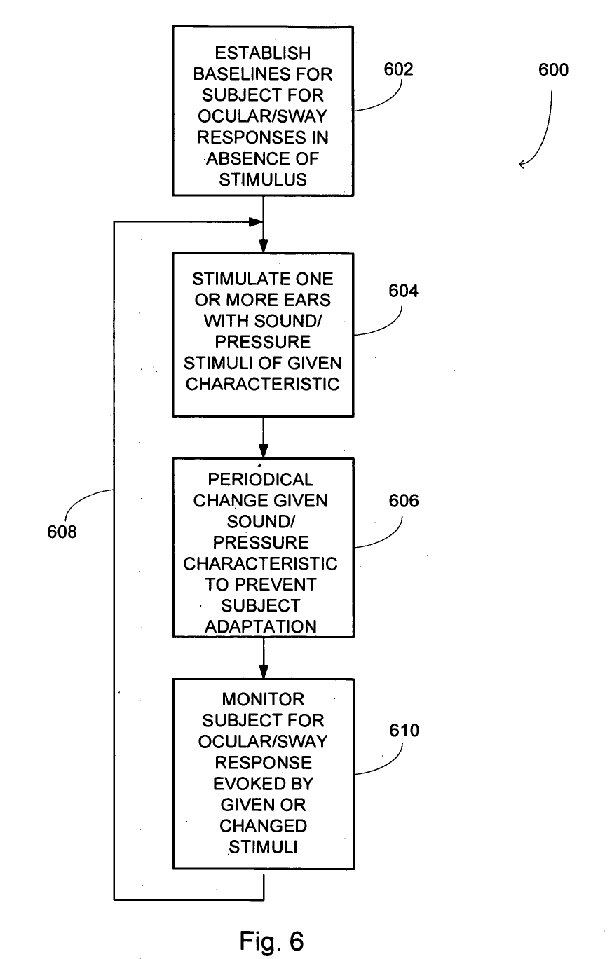

FIG. 6 illustrates the invented method 600 of increasing the accuracy of vestibular disorder detection and treatment. Those of skill will appreciate that, before the stimulating step, in accordance with certain embodiments of the invention, one or more baselines first are established at 602 for the one or more ocular and sway responses in the absence of stimulus. Thus, the monitoring in accordance with the invented method of increasing vestibular disorder testing is for one or more of an ocular and sway response outside the established one or more baselines. The defined amount, e.g. zero or more, by which the subject's measured ocular or sway response is outside the subject's established baseline (and/or is outside accepted statistical population norms, if desired) readily is determined by the ocular and sway response modules—e.g. by nystagmus analyzer 20 and head/torso motion analyzer 22 or software residing in computer 40 as part of expert subsystem 26—described and illustrated herein or can be determined by a live operator only partly reliant on the automated tools provided as a part of invented system 10.

The method further includes a) at 604, stimulating one or more of a subject's ears with sound and/or pressure stimuli of a given characteristic; and b) at 606 periodically changing the given characteristic of the sound and/or pressure stimuli and c) at 608 repeating the stimulating step in accordance with the changed characteristic, thereby to prevent adaptation by the subject to the stimuli Steps 604 and 606 (along with the looping or repeating step 608) are performed while c) at 610 monitoring the subject for one or more of an ocular and sway response evoked by the given or changed stimuli. In accordance with one embodiment of the invention, at least the stimulating and the monitoring steps 604 and 606 (along with the looping or repeating step 608) are computer-assisted, e.g. accomplished by executing in a suitable processor instructions that stored in memory. In this manner, the invented method is at least semi-automated.

In accordance with alternative embodiments of the invention, the periodic changing is computer-assisted and thus based upon a predefined anti-adaptation protocol, or it is manual and thus based upon a live operator's observations of adaptation by the subject. In any event, such changes are designed to reduce predictability by the subject of the stimuli. In other words, the changes are intended to take the subject by surprise so that the subject does not falsely test negative. In accordance with one embodiment of the invention, the characteristic that is changed unpredictably is tonal and the change itself is of one or more of the frequency and volume of the tonal characteristic. In accordance with another embodiment of the invention, the characteristic that is changed unpredictably is tonal and the change itself involves masking a given tonal characteristic by the superposition of another tone, e.g. producing a tone-on-tone or narrow-band noise intended to mask the given tone.

In accordance with one embodiment of the invention in which the subject is equipped with bilateral ear probes, the stimuli impact both ears of the subject. By virtue of this feature of the invention, the given tone can be made to impact one ear and the other tone can impact a contralateral ear. Thus the superposition of another tone can be in a given ear of the subject, or in accordance with another aspect of the invention can be in both ears of the subject. In either case, effective sound and/or pressure stimuli masking is possible that increases the accuracy of subject vestibular disorder testing by preventing subject adaptation. This is yet another advantage of the novel provision in accordance with the invention of (two) bilateral ear probes, one for each subject test ear.

It will be understood that the present invention is not limited to the method or detail of construction, fabrication, material, application or use described and illustrated herein. Indeed, any suitable variation of fabrication, use, or application is contemplated as an alternative embodiment, and thus is within the spirit and scope, of the invention.

From the foregoing, those of skill in the art will appreciate that several advantages of the present invention include provision of an integrated, all-in-one, expert vestibular function assessment tool that is easy and inexpensive to use and that can be easily installed for clinical use of transported and mobilized for field use. By providing sound and/or pressure stimuli in one or both subject ears and by monitoring the subject's sway and/or nystagmus response thereto, vestibular disorder can readily and semi-automatically tested, detected and/or diagnosed in a systematic manner that leads to more accurate detection and far fewer false-positive or false-negative indications. Importantly, in accordance with the invention, raw and at least partially processed data are gathered for each subject, presented to an operator for decision making in helpful representational form and recorded for further evaluation and/or treatment.

It is further intended that any other embodiments of the present invention that result from any changes in application or method of use or operation, method of manufacture, shape, size, or material which are not specified within the detailed written description or illustrations contained herein yet are considered apparent or obvious to one skilled in the art are within the scope of the present invention.

Finally, those of skill in the art will appreciate that the invented method, system and apparatus described and illustrated herein may be implemented in software, firmware or hardware, or any suitable combination thereof Preferably, the method system and apparatus are implemented in a combination of the three, for purposes of low cost and flexibility. Thus, those of skill in the art will appreciate that the method, system and apparatus of the invention may be implemented by a computer or microprocessor process in which instructions are executed, the instructions being stored for execution on a computer-readable medium and being executed by any suitable instruction processor.

Accordingly, while the present invention has been shown and described with reference to the foregoing embodiments of the invented apparatus, it will be apparent to those skilled in the art that other changes in form and detail may be made therein without departing from the spirit and scope of the invention as defined in the appended claims.

We claim:

1. Apparatus for testing a subject's vestibular response to ear pressure or sound stimuli, the apparatus comprising:
a headset including bilateral ear-worn devices;
a stimulus-producing mechanism operatively coupled with the headset configured to deliver defined air pressure or sound stimuli to the bilateral ear-worn devices; and
one or more of an ocular response analyzer and a sway response analyzer operatively coupled to the headset, the ocular response analyzer detecting the subject's ocular response to the stimuli by electronic means and the sway response analyzer detecting the subject's sway response to the stimuli by electronic means.

2. The apparatus of claim 1, wherein the apparatus comprises both the ocular response analyzer and a sway response analyzer.

3. The apparatus of claim 2, wherein the sway response analyzer includes a sensor of the subject's head or torso positional and orientation response to the stimuli and computerized comparison of potentially aberrational positional and orientational response sensor data from the subject to recorded baseline subject data.

4. The apparatus of claim 2 which further comprises:
a destabilizing device for reducing baseline postural stability in the subject.

5. The apparatus of claim 4 which further comprises:
a carrying case configured and dimensioned portably to contain and transport the headset including the ear-worn devices, the stimulus-producing mechanism, the ocular response analyzer, the sway response analyzer and the destabilizing device therein.

6. The apparatus of claim 1, wherein the bilateral ear-worn devices each include a probe configured to be inserted into a subject's ear in an operational alignment with the subject's ear canal and in sealing engagement with the wall of the subject's ear canal.

7. The apparatus of claim 6, wherein the headset includes a spring member configured automatically to urge the probes into the subject's corresponding external auditory canals under conditions of operational alignment with the external auditory canals and of sealing engagement with the walls of the external auditory canals.

8. The apparatus of claim 6, wherein the stimulus-producing mechanism is configured to deliver both defined pressure stimuli and defined sound stimuli to the bilateral ear-worn devices.

9. The apparatus of claim 1, wherein the bilateral ear-worn devices include headphones configured to fit over a subject's contralateral ears.

10. Apparatus for testing a subject's vestibular response to ear pressure or sound stimuli the apparatus comprising:
a headset including one or more ear-worn devices, each ear-worn device configured to be acoustically coupled with a subject's ear,
a stimulus-producing mechanism operatively coupled with the headset configured to deliver defined air pressure or sound stimuli to the one or more ear-worn devices; and
an ocular response analyzer operatively coupled to the headset, the ocular response analyzer detecting the subject's ocular response to the stimuli by electronic means.

11. The apparatus of claim 10, wherein the stimulus-producing mechanism is configured to deliver both defined pressure stimuli and defined sound stimuli to the one or more ear-worn devices.

12. The apparatus of claim 11, wherein the detecting of the subject's ocular response to the stimuli is further by computerized comparison of potentially aberrational response data from the subject to recorded baseline subject data.

13. The apparatus of claim 11, wherein the electronic means are videographic.

14. The apparatus of claim 12, wherein the one or more ear-worn devices include one or more probes, each probe configured to be inserted into a subject's ear in an operational alignment with the subject's ear canal and in a sealing engagement with the wall of the subject's ear canal.

15. The apparatus of claim 14, wherein each of the probes includes a corresponding microphone, which further comprises:
an external auditory canal seal analyzer operatively coupled with each microphone, the seal analyzer configured to detect and annunciate any substantial failure of sealing engagement between the corresponding probe and the wall of the subject's external auditory canal.

16. The apparatus of claim 15, wherein the headset includes a spring member configured automatically to urge the probes into the subject's corresponding external auditory canals under conditions of operational alignment with the external auditory canals and of sealing engagement with the walls of the external auditory canals.

17. The apparatus of claim 16, wherein each of the probes includes a corresponding tympanometric device for evaluating the condition of the subject's middle ear.

18. The apparatus of claim 17, wherein the probes are two in number and are bilaterally independently operable.

19. The apparatus of claim 17, wherein the probes are two in number and are bilaterally concurrently operable.

20. The apparatus of claim 19, wherein the probes are simultaneously operable.

21. The apparatus of claim 14 which further comprises:
a carrying case configured and dimensioned portably to contain and transport the headset including the one or more ear-worn devices, the stimulus-producing mechanism and the ocular response analyzer.

22. The apparatus of claim 10 which further comprises:
a sway response analyzer operatively coupled to the headset, the sway response analyzer including a sensor of the subject's head or torso positional and orientational response to the stimuli and computerized comparison of potentially aberrational positional and orientational response sensor data from the subject to recorded baseline subject data.

23. The apparatus of claim 22 which further comprises:
a destabilizing device for reducing baseline postural stability in the subject.

24. The apparatus of claim 23 which further comprises:
a carrying case configured and dimensioned portably to contain and transport the headset including the probes, the stimulus-producing mechanism, the sway response analyzer and the destabilizing device therein.

25. The apparatus of claim 11 which further comprises:
a sway response analyzer operatively coupled to the headset, the sway response analyzer including a sensor of the subject's head or torso positional and orientational response to the stimuli and computerized comparison of potentially aberrational positional and orientational response sensor data from the subject to recorded baseline inertial subject data.

26. The apparatus of claim 25 which further comprises:
a destabilizing device for reducing baseline postural stability in the subject.

27. The apparatus of claim 26 which further comprises:
a carrying case configured and dimensioned portably to contain and transport the headset including the ear-worn devices, the stimulus-producing mechanism, the ocular response analyzer, the sway response analyzer and the destabilizing device therein.

28. The apparatus of claim 10, wherein the ear-worn devices are two in number and are bilaterally independently operable.

29. The apparatus of claim 10, wherein the ear-worn devices are two in number and are bilaterally concurrently operable.

30. The apparatus of claim 29, wherein the ear-worn devices are bilaterally simultaneously operable.

31. The apparatus of claim 29 which further comprises:
an inclinometer configured to be worn by the subject, the inclinometer detecting a sway response of the subject to the defined pressure or sound stimuli.

32. The apparatus of claim 10, wherein the stimulus producing mechanism includes a threshold vestibular response level detector that automatically halts the stimulus producing mechanism when the threshold level is reached.

33. A method of testing a subject for response to pressure stimuli for purposes of vestibular condition detection, the method comprising:
equipping a subject with a headset including bilateral ear-worn devices, and
concurrently applying bilateral sound and/or pressure stimuli via the bilaterial ear-worn devices to one ear and to the contra-lateral ear.

34. The method of claim 33, wherein the pressure stimuli are sound pressure level (SPL) stimuli and represent one or more successive subject-recognizable sounds.

35. The method of claim 33, wherein the pressure stimuli are air pressure and represent one or more successive sharp-attack-puff pressure envelopes.

36. The method of claim 33 wherein the bilateral pressure stimuli are differential in polarity relative to a substantially atmospheric baseline.

37. The method of claim 33, wherein the applying is automated via the use of software programmed to execute out of a memory associated with a computer operatively coupled with the air pressure stimulus apparatus.

38. The method of claim 37, wherein the headset further includes bilateral ocular testing apparatus, which further comprises:
detecting a defined threshold level of nystagmus response of the subject to the pressure stimuli.

39. The method of claim 38, wherein the detecting is automated via the use of software programmed to execute out of a memory associated with a computer operatively coupled with the ocular testing apparatus.

40. The method of claim 33 which further comprises:
automatically halting the application of pressure stimuli upon the detecting of a defined threshold level of nystagmus response.

41. The method of claim 33, wherein the headset further includes sway response testing apparatus, which further comprises:
detecting a defined threshold level of sway response of the subject to the sound and/or pressure stimuli.

42. The method of claim 41, wherein the detecting is automated via the use of software programmed to execute out of a memory associated with a computer operatively coupled with the sway response apparatus.

43. The method of claim 42 which further comprises:
indicating a directional component of the sway response of the subject.

44. The method of claim 42, wherein the indicating includes a graphic visual display.

45. The method of claim 44 which further comprises:
automatically halting the application of pressure stimuli upon the detecting of a defined threshold level of sway response.

46. The method of claim 33 which prior to the applying further comprises:
encumbering the subject with a destabilizing device configured to undermine the subject's baseline postural stability.

47. A stimulus-evoked vestibular disorder evaluation system comprising:
a sound or aural pressure stimulus evocation apparatus affecting a subject's bilateral ear canals;
at least one of a postural and an ocular response measurement apparatus for analyzing the subject's vestibular response to a sound or aural pressure stimulus evoked by the sound or aural pressure stimulus evocation apparatus; and
a computer program in the form of instructions residing in a memory and executing in a processor, the instructions configured to control the sound or aural pressure stimulus evocation apparatus in accordance with a defined protocol and to monitor the at least one of the postural and the ocular response measurement apparatus in accordance with defined parameters.

48. The system of claim 47, wherein the computer program controls the sound or aural pressure stimulus evocation apparatus in automatic response to monitored parameters of the at least one of the postural and the ocular response measurement apparatus.

49. The system of claim 47, wherein the computer program presents analytic results representing the subject's sound or aural pressure stimulus and at least one of the responsive postural and ocular responses to an operator in display form.

50. The system of claim 49, wherein the display form consists of representations selected from one or more of text, numbers and graphics.

51. The system of claim 49, wherein the computer program presents instruction or guidance data to the operator.

52. The system of claim 47 which further comprises:
a subject archive configured to record one or more instances of the subject-specific sound or pressure stimulus and at least one of the postural and ocular responses over successive subject sessions.

53. The system of claim 47 which further comprises:
a display mechanism operatively coupled with the computer program, the display mechanism configured to present to an operator a graphical user interface (GUI) that includes a graphic representation of the subject's semicircular ear canals in spatial orientation corresponding with the subject's physical semicircular ear canals, the display mechanism configured further to visually highlight a candidate semicircular ear canal exhibiting a vestibular disorder based upon the subject's at least one of the postural and ocular responses to the sound or aural pressure stimulus.

54. The system of claim 53, wherein the display mechanism is configured further to present to an operator a graphic depiction of the subject's eyes in real-time representational response to actual real-time movement of the subject's eyes, and wherein the display mechanism is configured further to present to an operator graphic waveforms representative of such actual real-time movement of the subject's eyes.

55. The system of claim 54, wherein the display mechanism is configured further to present to an operator a graphic depiction of average movement of the subject's eyes over one or more defined periods of time.

56. The system of claim 55, wherein the display mechanism is configured further to present to an operator a graphic depiction of the subject's real-time postural sway represented by a sway trajectory trace overlayed on a defined sway grid.

57. The system of claim 56, wherein the display mechanism is configured further to present to an operator an indicator of one or more sound or aural pressure stimuli correlated in real time with the subject's response thereto.

58. The system of claim 47, wherein the defined response parameters comprehend baseline response parameters for the subject established in the absence of either sound or aural pressure stimulus.

59. The system of claim 47, wherein the defined protocol and the defined response parameters are stored in a memory.

60. The system of claim 47 which comprises an ocular response testing apparatus, wherein the ocular response testing apparatus includes one or more digital cameras trained on at least one of the subject's eyes to monitor the subject's ocular response to the sound or aural pressure stimulus.

61. The system of claim 60, wherein the ocular response testing apparatus includes two cameras each trained on one of the subject's eyes, and wherein the defined parameters include ocular response parameters indicative of ocular convergence.

62. The system of claim 47 which comprises a postural response testing apparatus, wherein the postural response testing apparatus includes a sensor configured to sense spatial orientation of one or more of the subject's head and torso.

63. The system of claim 47, wherein the stimulation evocation apparatus comprises:
a controller for providing control signals to a sound generator and to an air stream generator;
a sound generator responsive to the controller, the sound and pressure generator including for each of two bilateral channels:
a tone generator circuit, the tone generator configured to produce a tone of defined amplitude, frequency and tonal character,
a gain and/or attenuation circuit configured to provide gain or attenuation of the tone output by the tone generator circuit, and
a power amplifier circuit configured to amplify the gain and/or attenuated output of the gain and/or attenuation circuit; and
an air pressure generator responsive to the controller, the air pressure generator including for each of two bilateral channels:
an air compressor configured to produce a pulse stream of air at a given pressure and modulation,
and a filtering mechanism configured to filter the pulse stream of air produced by the air compressor selectively to smooth the pulse stream pressure and modulation therefrom; and
ear probes for the two bilateral channels, each ear probe operatively coupled with one of the sound generators and with one of the air pressure generators.

64. The system of claim 63, wherein each ear probe is further operatively coupled with an aural feedback mechanism for monitoring the two bilateral channels for at least one of sound pressure level (SPL) and air pressure content.

65. A method of increasing the accuracy of vestibular disorder detection and treatment, the method comprising:
stimulating one or more of a subject's ears with sound and/or pressure stimuli of a given characteristic; and
periodically changing the given characteristic of the sound and/or pressure stimuli and repeating the stimulating step in accordance with the changed characteristic, thereby to prevent adaptation by the subject to the stimuli, while
monitoring the subject for one or more of an ocular and sway response evoked by the given or changed stimuli,
at least the stimulating and the monitoring being computer-assisted.

66. The method of claim 65, wherein the periodic changing is computer-assisted and based upon a predefined anti-adaptation protocol.

67. The method of claim 66, wherein the given characteristic is changed in such manner as to reduce predictability by the subject of the stimuli.

68. The method of claim 67, wherein the given characteristic is tonal and involves a given tone and wherein the change involves masking a given tonal characteristic by the superposition of another tone.

69. The method of claim 68, wherein the stimuli impact both ears of the subject and wherein the given tone impacts one ear and wherein the other tone impacts a contralateral ear.

70. The method of claim 67, wherein the given characteristic is tonal and wherein the change is of one or more of the frequency and volume of the tonal characteristic.

71. The method of claim 65, wherein the periodic changing is manual based upon a live operator's observations of adaptation by the subject.

72. The method of claim 69, wherein the given characteristic is changed in such manner as to reduce predictability by the subject of the stimuli.

73. The method of claim 72, wherein the given characteristic is tonal and involves a given tone and wherein the change involves masking a given tonal characteristic by the superposition of another tone.

74. The method of claim 73, wherein the stimuli impact both ears of the subject and wherein the given tone impacts one ear and wherein the other tone impacts a contralateral ear.

75. The method of claim 65 which, before the stimulating, further comprises:
establishing one or more baselines for the one or more ocular and sway responses in the absence of stimulation, wherein
the monitoring is for one or more of an ocular and sway response outside the established one or more baselines a defined amount.
